(19) United States
(12) Patent Application Publication
(10) Pub. No.: US 2007/0269789 A1
(43) Pub. Date: Nov. 22, 2007

(54) Bioreactor for Producing a Tissue Prosthesis, Particularly a Heart Valve

(76) Inventors: Bruno Covelli, Suhr (CH); Rolf Mulli, Wohlen (CH)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/556,959

(22) PCT Filed: May 27, 2003
(86) PCT No.: PCT/EP03/05582

§ 371 Date: Jan. 31, 2007

(30) Foreign Application Priority Data
May 16, 2003 (DE) 10322024.0

(51) Int. Cl. A01N 1/02; C12M 3/00

(52) U.S. Cl. 435/1.1; 435/289.1

(57) ABSTRACT

The invention relates to a bioreactor for producing a tissue prosthesis, particularly a heart valve, comprising a reactor chamber for holding a fluid nutrient medium and a prosthesis support, a device for placing the prosthesis support in the area of the reaction chamber, and a drive device for generating a pulsation flow of the nutrient medium inside the reactor chamber. The bioreactor is characterized in that an exciter device is provided for generating a frequency excitation of the nutrient medium and/or of the prosthesis support, said frequency excitation superimposing the pulsation flow of the nutrient medium. The invention also relates to a method for operating a bioreactor.

The present invention relates to a bioreactor for producing a tissue prosthesis, in particular a heart valve, having a reactor chamber to hold a fluid nutrient medium and a prosthesis support, a device for placing the prosthesis support in the region of the reactor chamber and a drive device for generating a pulsation flow of the nutrient medium in the reactor chamber. The invention also relates to a method of operating a bioreactor.

This type of bioreactor and the associated method are known from DE 19919625 A1. In particular, the principles for the in-vitro production of a homologous heart valve are explained in detail in this document. Preferably, a prosthesis support which consists of a biodegradable support carrier material is colonised with the patient's own body cells, preferably with fibroblasts, myofibroblasts and/or endothelial cells. The prosthesis support specifies which vessels are to be cultivated therefrom. In particular, this technique is suitable for growing heart valves. However, before a colonised prosthesis support can be implanted as a replacement part in the body, the cells which have colonised the prosthesis support must form a stable matrix which is mechanically load-bearing and correspondingly must form a connective tissue-like structure. Such stable groups of cells for vessels and heart valves can be produced in the aforementioned bioreactors. In these bioreactors, a nutrient fluid for the cells flows in pulses around the colonised prosthesis support. The shearing forces of the flow pulses on the cell surfaces cause the cells to form a stable, mechanically load-bearing group of cells on the support. The structure of this artificially grown connective tissue not only contains the applied fibroblasts and endothelial cells but also the essential components of a normal matrix such as collagen, elastin and glycosaminoglycanes.

In the case of the known bioreactors, pulse frequencies are utilised to generate the pulsation flow similar to the heart frequency between 30-150 pulses/min. These bioreactors use an elastic wall or membrane which preferably forms the base of the reactor chamber. A mechanical drive or pneumatic pressure changes are used to impart a pulse-like movement to the elastic wall or the membrane, as a result of which the nutrient medium in the reactor chamber flows accordingly around the artificial tissue. The structure and the principle of this type of drive are described in detail in the aforementioned document.

Therefore, in order to increase the pulse frequency, it is also possible to increase the delivery quantity and as a consequence it is possible to influence the shearing forces upon the artificial tissue. This method principle is tried and tested and is already being utilised successfully at the present time. Nevertheless, efforts are also being made to bring about improvements in this field, in particular with regard to a more beneficial colonisation of cells, in generating a stabile matrix.

Therefore, it is the object of the present invention to provide a bioreactor and a method of operating the bioreactor which bring about an improvement in the growth of a tissue prosthesis.

In the case of a bioreactor of the type stated in the introduction, this object is achieved by virtue of the fact that an exciter device is provided for the purpose of generating a frequency excitation of the nutrient medium and/or of the prosthesis support, which frequency excitation is superimposed upon the pulsation flow of the nutrient medium.

Accordingly, the invention is based upon the hitherto known bioreactor which is able to transmit a heart-like pulse frequency to the nutrient medium via one or several elastic membranes. In addition, a superimposed frequency is imparted to the nutrient medium and/or the prosthesis support. This frequency excitation should encourage the cells (in particular undifferentiated cells, such as progenitor and stem cells) on the prosthesis support to achieve increased growth, differentiation and a stabile matrix formation. In a fluidic analysis of the procedures between nutrient fluid and the cell matrix, the inventors have actually recognised that the highly turbulent flow pulses tear open the boundary layer of the nutrient fluid on the cell matrix and stress the cells on the one hand mechanically at frequencies other than the frequency of the pulsating nutrient medium and on the other hand produce a more effective metabolic exchange. Therefore, a frequency other than the pulse frequency is imparted to the pulsating nutrient medium, in order in this way to enhance the aforementioned effects. Assessments demonstrate that with the optimum selection of these other frequencies, resonance phenomena can also occur in the group of cells which encourage particular activity in the cells. Where the term “prosthesis support” is used in this Application, it should also be taken into consideration that the prosthesis support can already be colonised by cells and that in some exemplified embodiments the prosthesis support decomposes as intended and at an advanced stage of culturing the procedures described in this case are also performed directly on the tissue prosthesis or at a preliminary stage thereof.

In accordance with an advantageous variation of the bioreactor, it is provided that the exciter device is configured in an adjustable manner with regard to its exciter frequency. The exciter frequency could also be configured adjustably with regard to its amplitude. In dependence upon the most varied parameters, it is possible by adjusting the exciter frequency to set the optimum operating point for the culturing of the cells. As already mentioned above, resonance phenomena can encourage particular activity in the cells. The adjustability assists in the optimum generation of resonance phenomena of this type.

Preferably, the exciter device can comprise an electromagnetic frequency generator. Tests have demonstrated that in particular frequencies should be used which are higher than the frequency of the pulsation flow of the nutrient medium. Such frequencies can be achieved particularly effectively using electromagnetic frequency generators. Furthermore, an electromagnetic frequency generator can be adjusted precisely to the desired frequency using cost-effective means.

Furthermore, the exciter device can comprise at least one sound probe which is disposed in the reactor chamber. This makes it possible to encourage the cells in an acoustic sound field on the support material to achieve increased growth, differentiation and a more stable matrix formation. The sound is preferably introduced into the nutrient medium and is thus transmitted indirectly to the prosthesis support.

In order to increase the functional integrity of the exciter device, this device can comprise at least one drive unit which is disposed outside the reactor chamber. Therefore, the drive unit is not subjected to the fluid nutrient medium. Owing to the fact that the bioreactor is preferably disposed in an incubator, the at least one drive unit can also be disposed outside the incubator, so that the drive unit does not encounter any heating problems.

In an advantageous embodiment of the at least one drive unit and the at least one sound probe, it is provided that a membrane is disposed as a coupling member between these two units, wherein the membrane closes a window region in the reactor chamber. This provides a fluid-tight separation between the sound probe and the drive unit.

In accordance with a further embodiment, the exciter device can also be configured in such a manner as to transmit a lengthening and/or compressive movement to the prosthesis support. In this regard, the device for placing the prosthesis support could also be formed accordingly for the purpose of performing these types of movement. These lengthening and/or compressive movements which are superimposed upon the pulsation flow of the nutrient medium enhance the aforementioned effects which serve to improve culturing.

The method of operating a bioreactor for the production of a tissue prosthesis, in particular a heart valve, is characterised by the steps of:

placing a prosthesis support in a reactor chamber,

generating a pulsation flow of a fluid nutrient medium located in the reactor chamber,

generating a frequency excitation of the nutrient medium and/or of the prosthesis support,

which frequency excitation is superimposed upon the pulsation flow.

In accordance with one method variation, it is also advantageous if the frequency of the pulsation flow is lower than the frequency of the superimposed frequency excitation. In particular, higher frequencies of the superimposed frequency excitation ensure that the boundary layer of the nutrient fluid on the cell matrix is torn open, as a result of which the cells are stressed mechanically at higher frequencies. Furthermore, this also produces an improved metabolic exchange.

In this case, frequencies in the pulsation flow in the range between 0 and 300 pulses/min. and frequencies in the superimposed frequency excitation in the range between 0 and 30 KHz have proven to be advantageous. The superimposed frequency excitation can be performed in this case by means of electrical, electro-mechanical or mechanical forces.

One method variation which has proven to be particularly convenient is where sound waves are introduced as the superimposed frequency excitation into the nutrient fluid. The sound waves can be introduced in such a manner that the cells are located in an acoustic sound field.

One further way of achieving the superimposed frequency excitation is to repeatedly lengthen and/or stretch the prosthesis support. This lengthening and/or stretching can also be performed by the indirect excitation via the nutrient medium and thus enhance the effect still further.

It has proven to be a further advantage if a variable pressure gradient is achieved over the prosthesis support or the tissue prosthesis. For this purpose, in the case of a further method variation it is provided that the placement of the prosthesis support in the reactor chamber and/or the manner in which the pulsation flow is generated in the fluid nutrient medium and/or the manner in which the superimposed frequency excitation is generated are accomplished in such a manner that that this pressure gradient occurs along the tissue wall. This effect also provides for greater excitation of the cells and thus serves to achieve a more stable matrix formation.

An embodiment of a bioreactor in accordance with the invention is described in detail hereinunder with reference to a drawing.

The single FIGURE shows a schematic illustration of a bioreactor in a full sectional view. The dimensions of the bioreactor are dependent upon the size of the tissue prosthesis which is to be produced and which later is to be implanted into the host body of the cells.

The bioreactor 1 illustrated in the FIGURE comprises a housing which is constructed substantially from three components. The housing components are a lower housing shell 2, a middle housing shell 3 disposed thereon and a reactor head 4 which is disposed on the middle housing shell 3. The housing shell 2 and the middle housing shell 3 are connected to each other at their edge regions by way of hinge connections 5 with an elastic membrane 6 inserted therebetween. The elastic membrane 6 causes a pulsation chamber 7 to be formed in the housing shell 2, said pulsation chamber being supplied with a drive medium (gas or fluid) via a connection line 8 which issues at the side into the housing shell 2. Located at the other end of the connection line 8 is a pulsation drive which is not described in detail here (see e.g. DE 19919625 A1). Formed above the elastic membrane 6 in the middle housing shell 3 is a preliminary reactor chamber 9 which is connected to a storage container, not illustrated in detail, for nutrient fluid via a supply line 10 which issues in at the side.

The reactor head 4 is placed at the top on to the middle housing shell 3 and is connected thereto by means of hinge connections 11. A seal, not illustrated in detail, provides a secure closure in the connection region. The reactor head 4 surrounds the main reactor chamber 12 of the housing.

A lower support device 13 is located in the transition region from the preliminary reactor chamber 9 and the main reactor chamber 12 and an upper support device 14 is located in the upper region of the reactor head 4. The schematically illustrated prosthesis support 15 is held between the lower support device 13 and the upper support device 14.

Located on the upper side of the reactor head 4 is a discharge line 16, via which the nutrient fluid can be discharged into the storage container, so that a substantially closed circuit is produced between the supply line 10 and the discharge line 16.

Disposed in each case on both sides of the reactor head 4 is a window 17, whose opening is closed in each case by an elastic membrane 18. On the side of the membranes 18 facing the main reactor chamber 12 there is located in each case an antenna or a probe 19. The probes 19 extend into the main reactor chamber 12 and extend at least partially at a spaced interval along the prosthesis support 15. Disposed on the side of the membrane 18 remote from the probe 19 are sound conductors 20 which are each connected to a frequency transmitter. In the present case, the excitation is performed via the frequency transmitters by means of electromagnetic forces. However, purely electrical or mechanical forces could also be utilised.

The lower support device 13 and the upper support device 14 are configured in such a manner as to also be able to exert a lengthening and compressive movement upon the prosthesis support 15.

The mode of operation and function of the bioreactor 1 described above will be explained in detail hereinunder.

With respect to the production of the tissue prosthesis and the further method steps required for this purpose, reference is made to DE 19919625 A1. The following description thus relates mainly to the mode of function of the bioreactor.

A pressure medium, in present exemplified embodiment a fluid, is introduced into the pulsation chamber 7 via the connection line. This medium flows in and out of the pulsation chamber 7 via the connection line, so that by virtue of the elastic membrane 6 which thus moves in a reciprocating manner, pulsation frequencies between 0 to 300 pulses/min. can be generated. The pulsation of the elastic membrane 6 is transmitted to the preliminary reactor chamber 9 which is filled with nutrient fluid. The pulse form is generally sinusoidal, but it has been shown that pulse forms in the manner of a heart beat have a stabilising effect upon some artificial cardiovascular tissues. Therefore, the drive unit (pump), not illustrated, can also generate a non-sinusoidal pulsation.

The pulsation of the medium is imparted to the nutrient fluid in the pulsation chamber 7 by means of this drive. An arrangement of suitable non-return valves in the supply line 10 and in the discharge line 16 enables the nutrient fluid to be delivered in a pulsed manner from the preliminary reactor chamber 9 into the main reactor chamber 12. The lower support device 13 is configured in such a manner that this flow can be achieved as a matter of course. The nutrient fluid flows through the main reactor chamber 12 and in so doing flows along the prosthesis support 15. At the upper end, any excess nutrient fluid leaves the main reactor chamber 12 via the discharge line 16.

In principle, the flow of the nutrient medium in the preliminary reactor chamber 9 and the main reactor chamber 12 can be adjusted in two operating modes.

1. The supply line 10 for the preliminary reactor chamber 9 is closed and the discharge line 16 from the reactor head 4 is open. As a consequence, the nutrient fluid is delivered in a pulsed manner in an upwards and downwards movement around the prosthesis support 15 or the artificial tissue.

2. The said valves which prevent any back flow are fitted in the supply line 10 and in the discharge line 16. As a consequence, the nutrient fluid is delivered in a pulsed manner without any back flow around the prosthesis support 15 or the artificial tissue. The nutrient fluid is thus delivered from a storage container, not illustrated, through the supply line 10 into the bioreactor 1 from where it is delivered via the discharge line 16 into a collection container (or the storage container).

Alternately, it is also possible to combine the two operating modes.

While the nutrient fluid flows past the prosthesis support 15 or the artificial tissue, the frequency transmitters 21 excite the probes 19 in the main reactor chamber 12 via the sound conductors 20. The frequency can be set in the range of 0 to several KHz. In the present exemplified embodiment, electromagnetic frequency transmitters 21, similar to drive units of acoustic loudspeakers, are utilised. As a consequence, a superimposed frequency excitation of the nutrient fluid is generated in the main reactor chamber 12 which has the positive effect for the culturing of cells as described above. The probes 19 can be configured in various ways. In principle, it is also possible to dispose this probe in the form a sleeve in an annular manner around the prosthesis support 15 or the artificial tissue.

The lower support device 13 and the upper support device 14 are configured in such a manner that they influence the flow of the nutrient fluid as little as possible. The geometry of these devices is tailored to suit the forms of the prosthesis support 15 or the artificial tissue. Preferably, a chemically inert synthetic material scaffold can be used as the support device 13 or 14 which is connected to the prosthesis support 15 or to the artificial tissue by way of clamps. The arrangement of the prosthesis support 15 or the artificial tissue in the main reactor chamber 12 and/or the type of pulse flow and/or the type of superimposed frequency excitation via the probes 19 can produce a pressure gradient across the wall of the prosthesis support 15 or the grown tissue with respect to the nutrient fluid.

The combinatory excitation effect upon the prosthesis support 15 or upon the artificial tissue in the main reactor chamber 12 leads to increased growth, differentiation and to a stable matrix formation, so that a durable tissue prosthesis is produced.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A bioreactor for the production of a tissue prosthesis, in particular a heart valve, said bioreactor comprising a reactor chamber for holding a fluid nutrient medium and a prosthesis support, a device for placing the prosthesis support in the region of the reactor chamber, and a drive device for generating a pulsation flow in the reactor chamber, said bioreactor further comprising an exciter device for generating a frequency excitation of the nutrient medium, of the prosthesis support, or of both, which frequency excitation is superimposed upon the pulsation flow of the nutrient medium.

15. The bioreactor as claimed in claim 14, wherein said exciter device is adjustable to thereby enable the generation of different exciter frequencies.

16. The bioreactor as claimed in claim 14, wherein said exciter device comprises an electromagnetic frequency generator.

17. The bioreactor as claimed in claim 16, wherein said exciter device comprises at least one sound probe provided within said reactor chamber.

18. The bioreactor as claimed in claim 14, wherein said exciter device comprises at least one drive unit disposed outside of said reactor chamber.

19. The bioreactor as claimed in claim 18, wherein said reactor chamber comprises at least one window region, said at least one window region being closed by a membrane, said membrane being configured as a coupling member between said at least one drive unit and at least one sound probe provided within said reactor chamber.

20. The bioreactor as claimed in claim 14, wherein said exciter device is configured to induce a lengthening movement, a compressive movement, or both a lengthening and compressive movement to said prosthesis support.

21. A method of operating a bioreactor for the production of a tissue prosthesis, in particular a heart valve, the method comprising:
placing a prosthesis support in a reactor chamber of said bioreactor;
generating a pulsation flow of a fluid nutrient medium located in said reactor chamber, and
generating a frequency excitation of said nutrient medium, of said prosthesis support, or of both, which frequency excitation is superimposed upon the pulsation flow.

22. The method as claimed in claim 21, wherein said generated pulsation flow and said generated frequency excitation each have a frequency, and said generated pulsation flow frequency is lower than said generated frequency excitation frequency.

23. The method as claimed in claim 22, wherein said pulsation flow frequency is less than or equal to 300 Hz and said generated frequency excitation frequency is less than or equal to 30 KHz.

24. The method as claimed in claim 21, wherein said frequency excitation comprises sound waves.

25. The method as claimed in claim 21, wherein as the superimposed frequency excitation, the prosthesis support is repeatedly lengthened, stretched, or both lengthened and stretched.

26. The method as claimed in claim 21, wherein one or more of the placement of said prosthesis support in said reactor chamber, the manner in which the pulsation flow is generated in the fluid nutrient medium, and the manner in which the superimposed frequency excitation is generated serve to generate a pressure gradient along the prosthesis support relative to the fluid nutrient medium.

27. A bioreactor for producing a prosthetic implant, the bioreactor comprising:
a reactor chamber having an internal cavity that defines a reservoir for a fluid nutrient medium;
a scaffold provided within said lumen, said scaffold being configured for supporting a biodegradable carrier for cellular material;
a driver operable at a first frequency to generate a pulsatile flow of nutrient medium in said reservoir; and
an exciter device operable to generate a frequency excitation at a second frequency different to said first frequency, said frequency excitation being superimposed on said pulsatile flow.

28. The bioreactor according to claim 27, wherein said second frequency is higher than said first frequency.

29. A bioreactor comprising:
a reactor vessel having an internal cavity, said internal cavity being divided by a flexible membrane into a pumping chamber and a reservoir chamber;
a fluid inlet/outlet opening to said pumping chamber to permit pumping fluid to enter into and be withdrawn from said pumping chamber to drive said flexible membrane and thereby establish a pulsatile flow of a first frequency in a fluid nutrient medium provided within said reservoir chamber,
a scaffold for supporting a biodegradable carrier within said reservoir chamber to thereby enable said pulsatile flow to mechanically stress said carrier;
one or more probes provided within said reservoir, and
one or more drivers each associated with a said probe, said drivers being operable to vibrate said probes at a second frequency higher than said first frequency and thereby vary the mechanical stress imparted to said carrier.
